(19) United States
(12) Patent Application Publication
(10) Pub. No.: US 2024/0420826 A1
(43) Pub. Date: Dec. 19, 2024

(54) SYSTEM AND METHOD OF RELAPSE PREDICTION AND ADDICTION TREATMENT SUPPORT USING A NEURAL NETWORK

(71) Applicant: Sensei AI Ltd, Modiin-Macabim-Reut (IL)

(72) Inventors: Ravit WEISMAN, Rishon Letzion (IL); Israel WEISMAN, Rishon Letzion (IL)

(73) Assignee: Sensei AI Ltd, Modiin-Macabim-Reut (IL)

(21) Appl. No.: 18/740,891

(22) Filed: Jun. 12, 2024

Related U.S. Application Data
(60) Provisional application No. 63/507,802, filed on Jun. 13, 2023.

(51) Int. Cl. G16H 20/70; A61B 5/0205; G16H 10/20; G16H 10/60; G16H 50/30; G16H 80/00

(52) U.S. Cl. CPC G16H 20/70; G16H 10/20; G16H 10/60; G16H 50/30; G16H 80/00; A61B 5/0205

(57) ABSTRACT

Methods and systems for predicting addiction relapse risk in a patient include; receiving patient data of a patient; predicting, by a trained neural network model, based on the patient data, an addiction relapse risk in the patient; generating a recommendation based on the predicted addiction relapse risk; and communicating the recommendation to a relevant party.

### FIELD OF THE INVENTION

The present invention relates generally to artificial intelligence and machine learning. More specifically, the present invention relates to neural network algorithms to predict and/or prevent relapse in individuals undergoing addiction treatment by leveraging personal data in real-time and asynchronous.

### BACKGROUND

Addiction is a chronic disease characterized by compulsive drug-seeking behavior or substance use as part of physical addiction use, and/or types of behavior addictions such as addiction to gambling or to shopping. Addiction may be referred to as substance use disorder (SUD).

All types of addiction come with harmful consequences. Relapse is a common occurrence in addiction recovery, and it can be triggered by various factors, such as stress, exposure to drug-related cues, or social situations. Timely identification of these factors and implementation of appropriate interventions can help reduce the likelihood of relapse.

Existing methods for predicting relapse in individuals undergoing addiction treatment typically rely on manual reporting and data collection, which may not always be accurate, timely, or comprehensive. There is a need for a more efficient and automated approach to predict relapse and assist healthcare professionals in providing timely and effective support to individuals in addiction treatment, and for insight based interventions and enforcement.

### SUMMARY OF THE INVENTION

The present invention may address this need by providing a neural network algorithm for predicting relapse in individuals undergoing addiction treatment. In some embodiments, the algorithm may leverage personal data, including physiological, psychological, and environmental factors, to identify events or situations that may lead to relapse.

In such an event, the algorithm may offer a real-time intervention using a Generative Pre-trained Transformer (GPT) model. The algorithm can be integrated into various platforms, such as wearable devices, mobile applications, or telemedicine systems, to facilitate real-time monitoring and support.

According to some embodiments, there is provided a method, the method including: receiving patient data of a patient; predicting, by a trained neural network model, based on the patient data, an addiction relapse risk in the patient; generating a recommendation based on the predicted addiction relapse risk; and communicating the recommendation to a relevant party.

According to some embodiments, the patient data is received from one or more of: a wearable device; a mobile device; a mobile application; and a telemedicine system.

According to some embodiments the patient data includes one or more of: heart rate; heart rate variability; skin conductance; blood pressure; sleep patterns; activity level; mood; stress level; geographic location; a therapy session transcript; textual patient assessment; qualitative patient assessment; and quantitative patient assessment.

According to some embodiments, the method includes preprocessing the patient data, wherein the preprocessing includes one or more of: natural language processing (NLP); named entity detection (NER); and tokenization.

According to some embodiments, generating a recommendation based on the predicted addiction relapse risk includes generating the recommendation using generative artificial intelligence.

According to some embodiments, the recommendation includes one or more of: a request for additional patient data; an intervention; a stress management technique; and a personalized message.

According to some embodiments, the addiction relapse risk is categorized as none, low, or high, wherein if the addiction relapse risk is categorized as low, the method includes communicating the recommendation to the patient, wherein the recommendation includes a request for additional patient data, and wherein if the addiction relapse risk is categorized as high, the method includes communicating the recommendation to the patient and a therapist of the patient, wherein the recommendation includes a suggested intervention generated by a generative artificial intelligence, and wherein optionally the suggested intervention includes input from the therapist.

According to some embodiments there is provided a system including: at least one computer processor; and a memory containing instructions which, when executed by the at least one computer processor, cause the at least one computer processor to: receive patient data of a patient; execute a neural network model trained to predict, based on the patient data, an addiction relapse risk in the patient; generate a recommendation based on the predicted addiction relapse risk; and communicate the recommendation to a relevant party.

According to some embodiments, the patient data is received from one or more of: a wearable device; a mobile device; a mobile application; and a telemedicine system.

According to some embodiments, the patient data includes one or more of: heart rate; heart rate variability; skin conductance; blood pressure; sleep patterns; activity level; mood; stress level; geographic location; a therapy session transcript; textual patient assessment; qualitative patient assessment; and quantitative patient assessment.

According to some embodiments, the at least one computer processor is configured to preprocess the patient data, wherein the preprocessing includes one or more of: natural language processing (NLP); named entity detection (NER); and tokenization.

According to some embodiments, the at least one computer processor is configured to generate a recommendation based on the predicted addiction relapse risk by generating the recommendation using generative artificial intelligence.

According to some embodiments, the recommendation includes one or more of: a request for additional patient data; an intervention; a stress management technique; and a personalized message.

According to some embodiments, the at least one computer processor is configured to categorize the addiction relapse risk as none, low, or high, wherein if the addiction relapse risk is categorized as low, the at least one computer processor is configured to communicate the recommendation to the patient, wherein the recommendation includes a request for additional patient data, and wherein if the addiction relapse risk is categorized as high, the at least one computer processor is configured to communicate the recommendation to the patient and a therapist of the patient, wherein the recommendation includes a suggested intervention generated by a generative artificial intelligence, and wherein the suggested intervention optionally includes input from the therapist.

According to some embodiments there is provided a method for predicting relapse in a patient, the method including: training a neural network model on patient data relating to a plurality of patients; predicting, by the trained neural network model, based on a set of runtime patient data, a relapse risk of the patient; recommending an action based on the predicted relapse risk; and notifying the patient or a predefined contact of the predicted relapse risk and recommended action.

According to some embodiments, the patient data relating to the plurality of patients and/or the runtime patient data are collected from one or more of: a wearable device; a mobile device; a mobile application; and a telemedicine system.

According to some embodiments, the patient data relating to the plurality of patients and/or the runtime patient data include one or more of: heart rate; heart rate variability; skin conductance; blood pressure; sleep patterns; activity level; mood; stress level; geographic location; a therapy session transcript; textual patient assessment; qualitative patient assessment; and quantitative patient assessment.

According to some embodiments, the method includes preprocessing the patient data relating to the plurality of patients and the runtime patient data, wherein the preprocessing includes one or more of: natural language processing (NLP); named entity detection (NER); and tokenization.

According to some embodiments, the preprocessing is used to extract features relevant to relapse risk contexts and correspondingly label the patient data relating to the plurality of patients for training the neural network model.

According to some embodiments, recommending an action based on the predicted relapse risk includes generating the recommended action using generative artificial intelligence.

According to some embodiments, a neural network algorithm for predicting relapse in individuals undergoing addiction treatment is provided. The neural network algorithm can include:

1. A data collection module configured to receive and preprocess personal data from various sources, such as wearable devices, mobile applications, or electronic health records.

2. A feature extraction module for identifying relevant features from the preprocessed data, including physiological signals (e.g., heart rate, skin conductance), psychological factors (e.g., mood, stress levels), and environmental factors (e.g., location, social interactions).

3. A neural network model includes multiple layers, such as input, hidden, and output layers, for processing the extracted features and predicting the likelihood of relapse.

4. A decision support module for generating personalized recommendations or interventions based on the predicted relapse risk, which may be communicated to the individual, healthcare professionals, or caregivers for timely action.

5. An evaluation module for assessing the performance of the neural network model and updating the model parameters based on feedback from the decision support module, thereby facilitating continuous learning and improvement.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic block diagram of an example computing device, according to some embodiments of the invention;

FIG. 2 is a schematic system diagram, according to some embodiments of the invention;

FIG. 3 is a system architecture showing steps in the processing by the system architecture, according to some embodiments of the invention;

FIG. 4 is a flowchart of a method, according to some embodiments of the invention;

FIG. 5 is a schematic flow diagram of an example processing of user input, according to some embodiments of the invention;

FIGS. 6A and 6B illustrate example views of a patient mobile device application for collecting data, according to some embodiments of the invention;

FIG. 7 is a schematic flow diagram of an example model integration and processing flow, according to some embodiments of the invention; and

FIG. 8 shows example processing layers, according to some embodiments of the invention.

Embodiments of the invention are illustrated by way of example and not limitation in the figures of the accompanying drawings, in which like reference numerals indicate corresponding, analogous or similar elements.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof may occur or be performed simultaneously, at the same point in time, or concurrently.

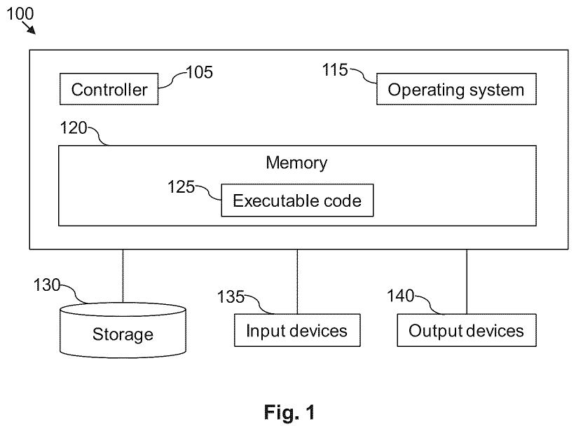

Reference is made to FIG. 1, which is a schematic block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. The computer network described herein may include one or more computing device(s) 100, for example, to act as the various devices or the components.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120. Storage 130 may include database storage. Storage 130 may include cloud-based storage.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device.

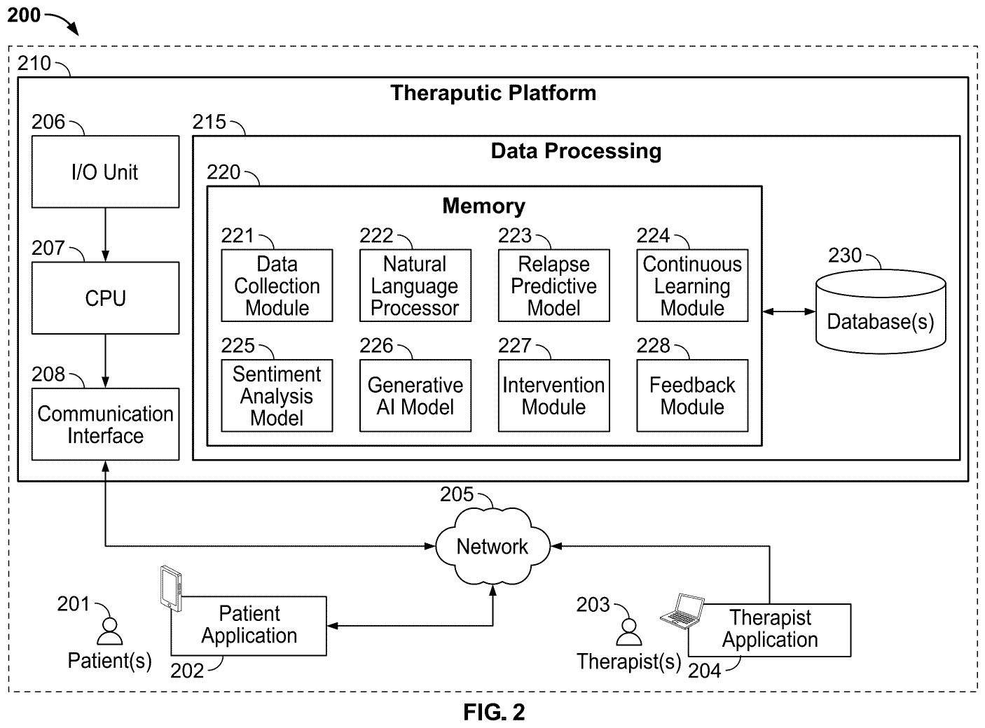

FIG. 2 shows a schematic diagram of a system 200, according to some embodiments of the invention.

System 200 may allow patient(s) 201 using patient application 202 and therapist(s) 203 using therapist application 204 to send and/or receive data over network 205 (e.g. a data communication network such as the internet) to a therapeutic platform 210.

Patient(s) 201 may be, for example, an individual undergoing treatment for addiction, such as an individual undergoing treatment for substance use disorder (SUD). Patient application 202 may run on, or otherwise be executed by, a device of patient(s) 201, such as a mobile device (e.g. smartphone), wearable device (e.g. smartwatch), laptop, or other computing device.

Therapist(s) 203 may be, for example, an individual providing treatment to patient(s) 201, such as treatment for addiction (e.g. treatment for SUD). Therapist application 204 may run on, or otherwise be executed by, a device of therapist(s) 203, such as a mobile device (e.g. smartphone), wearable device (e.g. smartwatch), laptop, or other computing device.

Therapeutic platform 210 may be, or may include elements of, a computing device, such as computing device 100 shown in FIG. 1. For example, therapeutic platform 210 may be, may be implemented by, or may include, a server.

Therapeutic platform 210 may include an input/output (I/O) unit 206. I/O unit 206 may be configured to input or output data, such as patient data of patient(s) 201.

Therapeutic platform 210 may include a computer processor unit (CPU) 207. CPU 207 may be the same as or similar to controller 105 described with respect to FIG. 1.

Therapeutic platform 210 may include a communication interface 208. Communication interface may allow communication between therapeutic platform 210 and network 205, for example to send/receive data to/from patient(s) 201 and therapist(s) 203.

Therapeutic platform 210 may include data processing module/unit 215. Data processing module/unit 215 may be a part of CPU 207. Data processing module/unit 215 may process data, such as patient data, for the purposes of carrying out methods according to embodiments of the invention.

Data processing unit 215 may include memory 220. Memory 220 may be the same or may be similar to memory 120 described with respect to FIG. 1. Memory 220 may contain executable instructions and/or code for carrying out methods according to embodiments of the invention. For example, memory 220 may include one or more of: data collection module 221; natural language processor 222; relapse predictive model 223; continuous learning module 224; sentiment analysis model 225; generative AI model 226; intervention module 227; and/or feedback module 228.

Data collection module 221 may be configured to receive patient data of a patient. The patient data may be received from one or more of: a wearable device (e.g. a smartwatch, fitness tracker, or the like); a mobile device; a mobile application (such as patient application 202 and/or therapist application 204); and/or a telemedicine system. In some embodiments of the present invention, data is leveraged from such personal electronic devices to collect information on the individual's mood, stress levels, and social interactions.

For example, patient data of a patient may include one or more of: heart rate; heart rate variability; skin conductance (e.g. galvanic skin response); blood pressure; sleep patterns; activity level; mood; stress level; geographic location; a therapy session transcript; textual patient assessment; qualitative patient assessment; and/or quantitative patient assessment.

Textual patient assessment may include a written description of how the patient is feeling, for example as provided by a patient in response to a prompt delivered by patient application 202 such as “How are you feeling today?”. The textual patient assessment may be analyzed according to processes described herein in order to gain an insight into the patient's emotional state or wellbeing, which may be indicators of addiction relapse risk.

A qualitative patient assessment may include a selection by the patient of a graphic symbol in patient application 202, such as a smiley face, sad face, thumbs up, thumbs down, or the like in order to convey in a qualitative sense the emotional wellbeing of the patient. A qualitative patient assessment may include a binary or Boolean value.

A quantitative patient assessment may include a value generated or calculated from one or more numerical values, such as heart rate and galvanic skin response according to one or more predefined formulas. A quantitative patient assessment may include a rating or score on a scale as input by a patient in response to a prompt from patient application 202. For example, a patient may be asked to rate how they are feeling on a scale from 1 to 10. A rating of 1 may indicate that the patient is feeling very negative, and a rating of 10 may indicate that the patient is feeling very positive. An intermediate value may characterize a patient wellbeing between the two extremes. Other values or mappings of values may be used (e.g. 1 being more positive, 10 being more negative).

In addition, embodiments of the invention may access electronic health records to incorporate relevant medical history and treatment progress.

In order to apply an effective data collection strategy, data collection module 221 may employ various data collection strategies, as described herein:

### Timely Data Collection

The timing of data collection can impact its value. Timely data collection may refer to collecting data at the relevant moments when it is mostly likely to yield a valuable insight. For example: the platform may prompt the patient to provide input before an exam to measure their anxiety or stress level, or following an exam to measure their mood.

### Data-Driven Data Collection

Data driven data collection involves using existing data to inform future data collection. For example, in embodiments where the system is using a NER (Named Entity Detection) algorithm for detecting that the patient is going on a date (e.g. a romantic date) by analyzing their chat conversation on the patient's mobile device application, it may prompt the patient to provide input shortly after the date to assess their mood and stress level.

Text Data from Interactions

The data from the patient's interaction with their therapist or support team on the mobile device application may be a rich source of information. For example, it might include message exchanges via the chat module on the mobile device application or therapist notes and summaries from in-person sessions. This data may be analyzed using natural language processing (NLP), such as by natural language processor 222, and preferably using NER to extract insights about the patient's emotional state, their thoughts, and their attitude toward their recovery. For example, if a patient writes using the chat support on the mobile device application 202 “I've been feeling really overwhelmed at work lately. I feel like I need a drink to cope,” the AI system may process the patient's message using Natural Language Processing. The NLP component may use tokenization, which breaks down the text into individual words or “tokens”. The sentence “I've been feeling really overwhelmed at work lately. I feel like I need a drink to cope.” might be tokenized into [′I′, ‘ve’, ‘been’, ‘feeling’, ‘really’, ‘overwhelmed’, ‘at’, ‘work’, ‘lately’, ‘I’, ‘I’, ‘feel’, ‘like’, ‘T’, ‘need’, ‘a’, ‘drink’, ‘to’, ‘cope’, ‘.’].

These tokens may then be passed through an embedding layer to be converted into a dense vector representation. Each token may be mapped to a vector in a high-dimensional space, where the location and distance (e.g. determined by a norm or metric) between vectors capture semantic meaning.

The sequence of vectors may then be fed into the predictive model, which in this case could be a Recurrent Neural Network (RNN) or a Transformer-based model like BERT (Bidirectional Encoder Representations from Transformers) or GPT (Generative Pre-Trained Transformations), trained to interpret the sequence and predict relapse risk.

The model may be trained on numerous examples of text data from recovering addicts, labeled with whether the user was at risk of relapse or not at the time of writing. It has learned to associate certain patterns of words and sentiments with increased relapse risk. For example, the model may have learned that phrases like “overwhelmed at work” or “need a drink to cope” are associated with higher relapse risk.

Active Input from Users

Active input from users may involve prompting the user to provide specific information or complete certain tasks. This might include answering daily check-in questions or surveys about their emotional state, stress levels, cravings, and other relevant factors.

For instance, the system might send a push notification to the user's mobile device each evening, asking them to rate their stress level and craving intensity for that day. This data can be fed into the AI system as quantitative data to supplement the qualitative insights from the text data.

### Passive Data Collection

Passive data collection refers to collecting data without requiring active input from the user. This might involve using wearable devices to continuously monitor the user's physiological parameters, such as heart rate, blood pressure, skin conductance (galvanic skin response) and sleep patterns.

By correlating these physiological markers with the user's self-reported stress levels and the text data from their interactions, the system can develop a comprehensive understanding by adding more these markers into the relapse predictive model as features. Adding these features may lead to more accurate predictions of the patient's risk of relapse.

Embodiments of the invention may further leverage data by integrating it into a wearable device, such as a smartwatch, to monitor an individual's physiological signals, including heart rate, skin conductance, and movement patterns. These devices may continuously monitor a wide array of vital signs, providing an objective, quantitative measure of a patient's physical state that can supplement the qualitative data gathered through text interactions and self-reports.

Some types of data which may be collected and processed by embodiments of the invention are now discussed in greater detail.

### Heart Rate

Continuous heart rate monitoring may help identify periods of high stress or anxiety, which may be linked to an increased risk of relapse. Abnormally high or rapidly fluctuating heart rates can be an indicator of acute stress.

### Heart Rate Variability (HRV)

HRV is the variation in the time interval between consecutive heartbeats. Lower HRV has been associated with stress, anxiety, and depression, so continuous monitoring of HRV may provide insights into the patient's emotional state.

### Blood Pressure

Elevated blood pressure can also be a sign of acute stress or anxiety. Tracking blood pressure changes in real-time may help to identify critical situations when an intervention might be required.

### Sleep Patterns

Quality of sleep can have a significant impact on mental health and the ability to cope with cravings and resist relapse. Wearable devices can track sleep duration, quality, and patterns, identifying issues like insomnia that might need to be addressed as part of the recovery process, and thus such data may be collected by embodiments of the invention.

### Activity Levels

Physical activity has a direct impact on mental health and well-being. Tracking a patient's daily activity can provide insights into their overall health and well-being, as well as their adherence to recommended exercise routines or other activities that may be part of their recovery plan. A patient not engaging with their recovery plan may be at higher risk of relapse.

### Galvanic Skin Response (GSR)

GSR measures changes (e.g. conductance changes) in sweat gland activity, which can indicate emotional stress, arousal, or excitement. An increase in GSR can be a sign of increased stress levels or emotional distress, and may indicate a relapse may be more likely.

Returning to FIG. 2, memory 220 may include a sentiment analysis model 225. Sentiment analysis module 225 may be configured to identify a sentiment in textual data, such as transcripts of therapy sessions, patient messages, and/or textual patient self-assessments to identify how the patient is feeling or what emotion the patient is conveying. Sentiment analysis model may be in operative communication with natural language processor 222 in order to analyze textual patient data.

Relapse predictive model 223 may be a neural network model or algorithm trained to predict, based on data collected by data collection module 221, an addiction relapse risk in a patient.

Embodiments of the invention may be trained on a large dataset of individuals undergoing addiction treatment, with information on relapse events and corresponding personal data. The model may utilize various machine learning techniques, such as deep learning, reinforcement learning, or unsupervised learning, to optimize prediction accuracy and adapt to the individual's unique circumstances.

The training dataset for this neural network model may be comprehensive and may include a variety of data points, such as:
Patient demographics—this may include age, gender, socioeconomic status, education level, and other demographic details.
Clinical data—This may include details of the substance or behavior the patient is addicted to, the duration of addiction, any prior attempts at recovery, co-occurring mental or physical health conditions, and other details.
Behavior data—this may include data related to the individual's behavior patterns, such as sleep, physical activity, and social interactions.
Textual data—this may include transcripts of therapy sessions, patient self-reports, and interaction with the AI system through the mobile device application.
Physiological data—this may include heart rate, blood pressure, sleep patterns, and other data collected from wearable devices.

Relapse predictive model 223 may be trained on such data using different training techniques, as described herein.

### Customized Model Training Based on Organizational Methodologies

A neural network system according to embodiments of the invention may be capable of segregating model training to align with the specific therapeutic approaches and methodologies of different organizations (e.g. organizations providing addiction therapy). This means that instead of using a one-size-fits-all model, embodiments of the invention allow for training a neural network to fit the distinct therapy methodologies of each organization. This customization may enhance the accuracy and relevance of predictions and automated interventions, ensuring that the AI supports the therapist's preferred treatment plans and techniques.

For example: a SUD rehabilitation organization with a unique therapy methodology that emphasizes Cognitive Behavioral Therapy (CBT) combined with mindfulness practices. The AI system may be adapted to this specific methodology to provide relevant and effective automated interventions and predictions.

For that purpose, embodiments of the invention may include collecting this data using different devices, mobile devices and input channels:

### 1. Patient Interaction Data:

### Text-Based Therapy Sessions:

Transcript: “I've been feeling very anxious lately, especially when I think about work. I can't seem to calm down.” Therapist response: “Let's focus on identifying the thought patterns that lead to your anxiety and practice mindfulness to help you manage these feelings.”

### Voice Notes:

Patient: “I noticed that my heart starts racing whenever I have to speak in a meeting.” Therapist: “Try taking deep breaths and grounding yourself in the present moment before the meeting.”

### 2. Patient Assessment Data:

### Self-Reported Surveys and Check-Ins:

Question: “On a scale of 1-10, how often do you feel anxious during the day?” Patient Response: “8”

Question: “Do you practice any mindfulness exercises?” Patient Response: “Yes, I meditate for 10 minutes every morning.”

### 3. Weekly Progress Reports:

“Patient shows a significant reduction in anxiety levels after incorporating daily mindfulness exercises.” “Cognitive restructuring techniques have helped the patient manage work-related stress more effectively.”

### 4. Therapy Session Data:

### Session Notes:

“Discussed the importance of recognizing and challenging negative thought patterns.” “Introduced mindfulness breathing exercises to manage anxiety.” “Patient practiced grounding techniques during the session and reported feeling more centered.”

### 5. Outcomes and Feedback:

### Follow-Up Surveys:

“Have you noticed an improvement in your anxiety levels since starting therapy?”, Patient Response: “Yes, I feel more in control of my thoughts and emotions.” “How helpful do you find the mindfulness exercises?”, Patient Response: “Very helpful, they make a significant difference.”

### The Customized Training Process:

Data Segmentation: Embodiments may include separating data related to CBT and mindfulness practices, and focusing on interactions and outcomes that align with the SUD rehabilitation organization methodology.

Training Data Preparation: Embodiments may include combining text, voice, and assessment data to create a comprehensive dataset. The data may be annotated (e.g. labeled) to highlight key aspects of CBT and mindfulness interventions.

Model Training: Embodiments may include training the neural network using the annotated dataset. Embodiments may include using transfer learning or similar methodology to adapt pre-trained models to the specific therapy methodology. The model may be validated against a test set to ensure it aligns with the SUD rehabilitation organization therapy approach.

Outcome: By customizing the model training process to fit the specific methodology of the SUD rehabilitation organization, an AI system according to embodiments of the invention can provide tailored interventions and predictions that are relevant to the particular SUD rehabilitation organization's unique approach. This ensures that the automated recommendations and insights are aligned with the therapeutic practices of the organization, enhancing the effectiveness of the treatment provided to the patients.

### Adaptive Training Algorithms

In some embodiments, training the neural network according to embodiments of the invention includes employing adaptive training algorithms that continuously learn and evolve based on real-time data from patient interactions and outcomes. Continuous learning module 224 may be involved in, or used for, this process. This adaptive learning capability may ensure that the model remains up to date with the latest treatment trends and patient responses, improving its predictive accuracy and intervention suggestions over time.

For example: A therapy center offers a blend of Dialectical Behavior Therapy (DBT) and art therapy. The adaptive training algorithms in embodiments of the invention may therefore need to continuously learn and evolve based on real-time data from patient interactions and therapy outcomes, ensuring the model remains relevant and effective.

For that purpose, embodiments of the invention may collect this data using its different devices, mobile devices and input channels:

### 1. Patient Interaction Data:

### Text-Based Therapy Sessions:

Transcript: “I'm struggling to manage my emotions, especially when I feel overwhelmed.” Therapist Response: “Let's use DBT techniques to help regulate your emotions. Have you tried journaling your thoughts?” Follow-Up Interaction: “Journaling helped a bit, but I still feel overwhelmed at times.”

### Voice Notes:

Patient: “Creating art helps me express my feelings, but I need more strategies for daily stress.” Therapist: “Art therapy is a great tool. Combine it with DBT skills like mindfulness to manage stress.”

### 2. Patient Assessment Data:

### Self-Reported Surveys:

Question: “On a scale of 1-10, how often do you feel overwhelmed?” Patient Response: “7”

Question: “How effective do you find art therapy in managing your emotions?” Patient Response: “Moderately effective.”

### Weekly Progress Reports:

“Patient shows improved emotional regulation through a combination of DBT and art therapy.” “Patient reports increased stress management after integrating mindfulness exercises.”

### 3. Therapy Session Data:

### Session Notes:

“Reviewed DBT techniques for emotional regulation.” “Encouraged the patient to explore different art mediums to express emotions.” “Practiced mindfulness exercises during the session.”

### 4. Outcomes and Feedback:

### Follow-Up Surveys:

“Have you noticed an improvement in your emotional regulation since starting therapy?” Patient Response: “Yes, especially with the DBT techniques.”

“How helpful do you find the combination of art therapy and DBT?” Patient Response: “Very helpful, it's a good balance for me.”

### Adaptive Training Process:

Continuous Data Collection: Embodiments may collect ongoing data from therapy sessions, patient assessments, and follow-up surveys. Embodiments may update the dataset with new interactions and outcomes regularly.

Real-Time Analysis: Embodiments may use real-time analysis to identify trends and patterns in patient responses and outcomes. Embodiments may adjust the weight of different features based on their impact on therapy effectiveness.

Model Retraining: Embodiments may include periodically retraining the neural network using the updated dataset and implementing transfer learning techniques to incorporate new data without forgetting previously learned information. The updated model may then be validated to ensure it maintains high accuracy and relevance.

Outcome: By employing adaptive training algorithms, embodiments of the invention may continuously learn from new data and adjust predictive models (e.g. relapse predictive model 223) to reflect the latest therapy trends and patient responses. This may ensure that the model remains up-to-date and provides relevant, effective interventions tailored to the unique therapeutic approach of the therapy provider. The adaptive learning capability may enhance the system's ability to deliver precise, personalized care that evolves with the needs of the patients.

### Hierarchical Model Training Architecture

In some embodiments of the invention, a hierarchical model training architecture is used that allows for multi-level training processes. This architecture may support the integration of general mental health treatment principles at a base level while enabling the incorporation of specific organizational methodologies at higher levels. This approach may ensure that the neural network can generalize well across diverse patient populations while still providing tailored interventions that align with the specific practices of each organization.

For example: consider a therapy organization that integrates general cognitive-behavioral principles with specific family therapy techniques. The hierarchical model training architecture in Sensei's system is designed to support multi-level training processes, allowing the integration of both general and specialized methodologies.

For that purpose, embodiments of the invention may collect this data using the different devices, mobile devices and input channels described herein:

### 1. Patient Interaction Data:

### Text-Based Therapy Sessions:

Transcript: “I've been feeling a lot of stress at home, especially with family conflicts.” Therapist Response: “Let's explore some cognitive-behavioral strategies to help manage your stress and then discuss how to improve communication with your family.”

### Voice Notes:

Patient: “Family dinners often end in arguments, and it's affecting my mental health.” Therapist: “We'll use some family therapy techniques to address these conflicts and build better communication skills.”

### 2. Patient Assessment Data:

### Self-Reported Surveys:

Question: “On a scale of 1-10, how stressed do you feel due to family conflicts?”

Patient Response: “8”

Question: “Have you tried any family therapy techniques to manage these conflicts?” Patient Response: “No, not yet.”

### Weekly Progress Reports:

“Patient shows reduced stress levels after applying cognitive-behavioral strategies.”

“Patient reports improved family interactions following family therapy sessions.”

### 3. Therapy Session Data:

### Session Notes:

“Reviewed cognitive-behavioral techniques for stress management.” “Introduced family therapy exercises to improve communication and resolve conflicts.”

“Practiced role-playing scenarios to address specific family issues.”

### 4. Outcomes and Feedback:

### Follow-Up Surveys:

“Have you noticed a reduction in stress since starting therapy?” Patient Response:

“Yes, the cognitive-behavioral techniques have been helpful.” “How effective do you find the family therapy sessions in improving communication?” Patient Response: “Very effective, we've had fewer arguments.”

### Hierarchical Model Training Process:

Base-Level Training: Embodiments may include training the neural network on general cognitive-behavioral therapy (CBT) principles using a broad dataset and ensuring the base model can generalize well across diverse patient populations.

Specialized Training Layers: Embodiments may include introducing additional training layers focused on specific family therapy techniques, and using data specific to family therapy interactions, including role-playing scenarios and communication exercises. These layers may be trained to fine-tune the model's understanding and recommendations for family therapy contexts.

Integrated Model Validation: Embodiments may include validating the hierarchical model using a combination of general CBT data and specialized family therapy data, and ensuring the model performs well in both general and specific therapeutic contexts.

Outcome: By utilizing a hierarchical model training architecture, embodiments of the invention system may effectively integrate general mental health treatment principles with specialized therapeutic techniques. This multi-level training approach may ensure that the neural network can generalize well across a wide range of therapeutic situations while providing precise, tailored interventions that align with the specific methodologies of the therapy organization. The hierarchical structure may allow the model to maintain high accuracy and relevance in both general cognitive-behavioral therapy and specialized family therapy, enhancing the overall effectiveness and personalization of the care provided.

### Data Segmentation and Reduction Techniques

To handle the vast amount of data generated in mental health and addiction therapy, embodiments of the invention may include the implementation of advanced data segmentation and reduction techniques. These techniques may ensure that only the most relevant and high-quality data is used for training the neural network, thereby enhancing the efficiency and performance of the model. By reducing noise and focusing on critical data points, embodiments of the invention may make more accurate predictions and provide more effective automated interventions.

For example: A therapy center that employs both mindfulness-based stress reduction (MBSR) and traditional psychotherapy. Embodiments of the invention may include data segmentation and reduction techniques to handle the vast amount of data generated, ensuring that only the most relevant and high-quality data is used for training the neural network.

For that purpose, embodiments of the invention may collect this data using the different devices, mobile devices and input channels as described herein and then process this data.

### Data Segmentation and Reduction Process:

Initial Data Segmentation: Embodiments may include separating data into categories such as mindfulness practices, cognitive restructuring, and general stress management. Embodiments may include identifying and tagging interactions, assessments, and outcomes specific to each category (e.g. annotating/labelling the data).

Relevance Filtering: Embodiments may include using algorithms to filter out less relevant data points, focusing on high-quality interactions that provide the most insight into therapy effectiveness, and prioritizing data that shows clear relationships between specific techniques (e.g., mindfulness) and patient outcomes.

Data Reduction Techniques: Embodiments may include applying dimensionality reduction techniques (e.g., Principal Component Analysis) to reduce the complexity of the dataset while retaining essential information, and using clustering algorithms to group similar data points, reducing redundancy and highlighting key patterns.

Training Data Preparation: Embodiments may include combining the segmented and reduced data into a streamlined dataset for model training, and ensuring the dataset includes a balanced representation of both MBSR and traditional psychotherapy techniques.

Outcome: By implementing advanced data segmentation and reduction techniques, embodiments of the invention may ensure that the training dataset is both relevant and high-quality. This process may eliminate noise, and focus on critical data points, enhancing the efficiency and performance of the neural network. The result may be a more accurate and effective model that can provide precise, tailored interventions for patients at the therapy center. This streamlined approach may allow the AI system to make better predictions and deliver more effective automated care suggestions, ultimately improving patient outcomes.

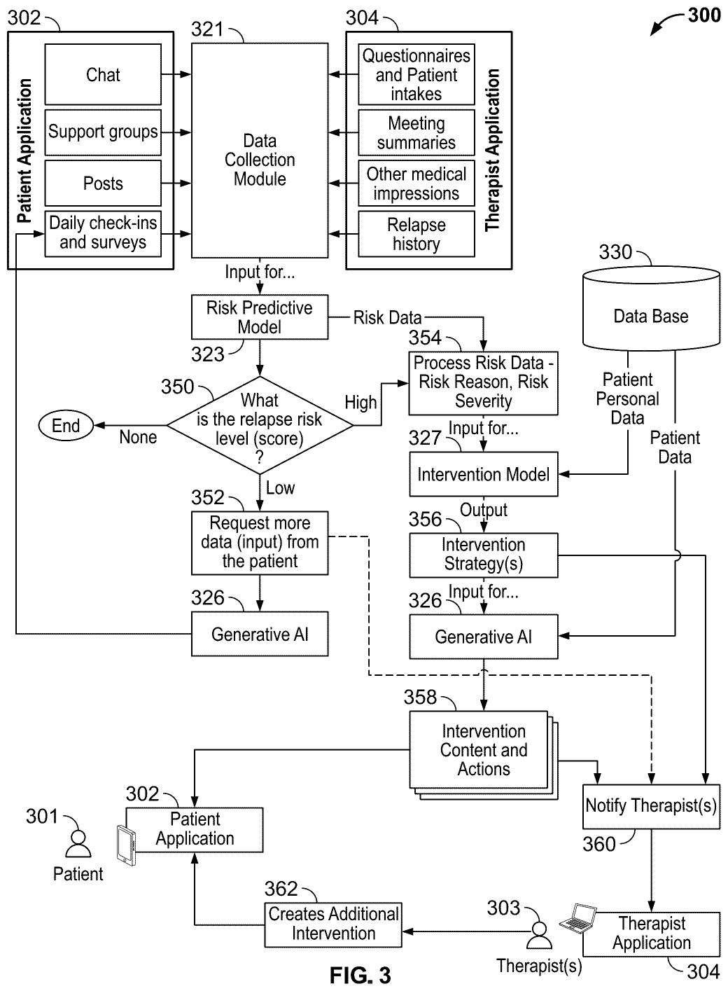

FIG. 3 is a system architecture 300 showing steps in the processing by the system architecture, according to some embodiments of the invention.

FIG. 3 shows some elements or modules as described in FIG. 2. For example, the flow of data in system 300 may start at data collection module 321, which may be as described for data collection module 221 shown in FIG. 2.

Data collection module 321 may receive data from patient application 302, and/or receive data from therapist application 304.

Data received from patient application 302 may include one or more of chat data, support group data, post data, and/or daily check in and survey data. For example, patient application 302 may be similar to a social media application where a patient can send messages (chats), post updates, and join online support groups.

Data received from therapist application 304 may include one or more of questionnaires and patient intakes, meeting summaries, other medical impressions (e.g. therapist notes), and/or relapse history.

Data collected by data collection module 321 may be used as input to risk predictive model 323. As described herein, risk predictive model 323 may be a trained neural network algorithm trained on historic past patient data and/or implementing continuous learning. Risk predictive model 323 may be trained using one or more techniques described herein, such as: customized model training based on organizational methodologies; adaptive training algorithms; and/or hierarchical model training architecture. Training risk predictive model 323 may include data segmentation and reduction techniques as described herein.

Risk predictive model 323 may determine (e.g. in a step 350) what is relapse risk level (e.g. quantified by a score or probability). In some embodiments, the addiction relapse risk is categorized as none, low, or high. For example a risk of “none” may include a probability of relapse of 0-10%, a risk of “low” may include a probability of relapse of 11-39%, and a risk of “high” may include a probability of 40-100%. Other probability ranges may be used.

If the addiction relapse risk is “none”, embodiments of the invention may take no further action. For example, a method or algorithm according to embodiments of the invention may end.

If the addiction relapse risk is “low”, embodiments of the invention may request more data from the patient (e.g. Step 352). The “low” categorization may be because there is insufficient data to make an assessment on the relapse risk of the patient, e.g. it may be early in the day or late in the day since a last check-in or survey was completed by the patient. In some embodiments, generative AI 326 may generate a request or prompt to be sent to a patient via patient application 302 requesting additional data, such as a further check-in or survey. In some embodiments, as part of Step 352, a therapist may be notified of the low risk of relapse.

If the addiction relapse risk is “high”, embodiments of the invention may process risk data, for example to identify the risk reason and/or the risk severity (e.g. Step 354). Risk data may be communicated from risk predictive model 323, which may be input to intervention model 327.

Intervention model 327 may analyze risk data and patient data (such as patient personal data obtained from database 330). Intervention model may output one or more intervention strategies 356, which may further be input to generative AI 326 to provide personalized recommendations, intervention content, and suggested actions 358. Such intervention content 358 may be automatically communicated to patient 301 via patient application 302. In some embodiments, the intervention content 358 may be notified to therapist 303 via therapist application 304 as part of Step 360. Therapist 303 may supplement or provide additional intervention content 362, which may then be communicated to patient 301.

The ability for a therapist to receive intervention content from generative AI 326 may reduce the time it takes for the therapist to create tailored intervention content for the patient, and thus increase the number of patients which the therapist can attend to within the same timeframe. For example, the same number of therapists may attend to a greater number of patients as a result of embodiments of the invention, compared to the number of patients without embodiments of the invention.

Embodiments of the invention may communicate intervention content or recommendations to one or more relevant parties. A relevant party may include the patient (e.g. patient 301) and the therapist (e.g. therapist 303), but may also include: one or more other therapists (e.g. as part of a therapy team for the patient); a predefined friend of the patient; a predefined family member of the patient; a caregiver of the patient; a sponsor of the patient; a care worker; a social worker; a support worker; a medical professional; and/or an emergency service (e.g. 911, ambulance), or the like.

An intervention may include an enforcement which may be carried out by embodiments of the invention. For example, an enforcement may be or may relate to one or more of: Creating geofencing based on the user's location; Automatically connect emergency service or contact person; providing incentives to encourage positive behavior (e.g. positive reinforcement); and/or real-time biofeedback intervention.

Some embodiments of the invention include using geofencing technology to monitor and control the patient's location in certain areas. For example, if a patient suffers from addiction and the mobile sensors detect that they enter a high-risk area (e.g. places associated with past substance use, a casino, or other place detrimental to addiction recovery), the system may lock specific functionalities on the patient's mobile device such as payments, social networks, messaging applications, and/or alert caregivers, send a reminder to leave the area, or the like. Additionally, integration with transportation services such as ride hailing apps can prevent the patient from ordering rides to these locations.

Some embodiments of the invention include automatically connecting to emergency services and/or a contact person (e.g. predefined relevant party such as a family member, friend, sponsor, etc.). For example, embodiments of the invention may include a feature to automatically contact emergency services or designated contacts when critical relapse or worsening in condition thresholds are detected, such as in cases of severe mental health deterioration or detected high relapse risk, the system can directly contact emergency services, provide location data, and share critical health information, ensuring rapid response and potentially stopping the deterioration or preventing further worsening of the condition.

Some embodiments of the invention include providing incentives to encourage positive behavior. For example, embodiments of the invention may use the AI algorithms and techniques described herein to track patient progress and behavior, providing dynamic incentives to encourage positive behavior and adherence to treatment plans. Embodiments of the invention may automatically reward patients with virtual tokens or points for completing certain tasks or reaching milestones (e.g. attending therapy sessions, taking medications on time, avoiding high-risk areas). These rewards can be redeemed for real-world benefits such as gift cards, discounts, or access to premium services. If the patient shows signs of relapse or non-compliance, the system may reduce or suspend these rewards.

As an example: for a patient who regularly attends scheduled therapy sessions and takes their medication on time the system tracks this behavior and rewards the patient with points. The patient can then redeem these points for a discount at a local grocery store. If the patient misses a session or a medication dose, the system temporarily suspends the rewards and alerts the caregiver.

Some embodiments of the invention may include real-time biofeedback intervention. Real-time may correspond to an event to system response on the order of milliseconds or microseconds. Embodiments of the invention may be integrated with mobile and wearable devices that monitor physiological markers (e.g. heart rate variability, skin conductance, and respiration rate) to detect early signs of stress, anxiety, or relapse. When the system detects abnormal physiological patterns indicative of stress or potential relapse or when the AI algorithms detects a worsening in condition or a risk for relapse, embodiments of the invention may provide real-time biofeedback interventions. These interventions can include guided breathing exercises, mindfulness activities, or cognitive-behavioral techniques delivered through the patient's mobile device or wearable. For example: The system detects increased skin conductance and irregular breathing patterns, indicating high-stress levels. It immediately sends a notification to the patient's smartwatch, guiding them through deep breathing exercises. The system monitors the patient's response to the intervention and adjusts the necessary exercises to help reduce stress.

Such interventions and enforcements as described herein may utilize the data collection and AI analysis techniques described herein to provide real-world effects for improving addiction recovery.

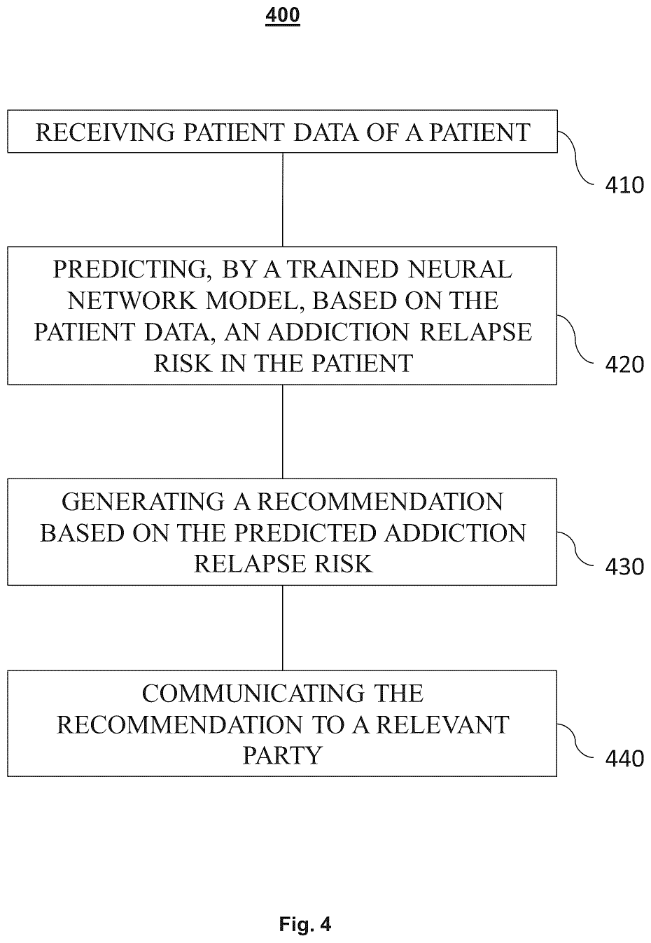

FIG. 4 is a flowchart of a method 400, according to some embodiments of the invention.

Method 400 may include receiving patient data of a patient (Step 410). Data may be received, or may be collected by, a module, such as data collection module 221 shown in FIG. 2. The patient data may be directly of the patient, e.g. a measured parameter of the patient such as heart rate, a patient submitted survey score, or indirectly of the patient, such as therapist notes relating to the patient. In some embodiments, the patient data is received from one or more of: a wearable device; a mobile device; a mobile application; and/or a telemedicine system. The patient data may be received from, for example, a patient application (such as patient application 202 shown in FIG. 2). The patient data may be received from, for example, a therapist application (such as therapist application 204 shown in FIG. 2).

In some embodiments, the patient data includes one or more of: heart rate; heart rate variability; skin conductance (e.g. galvanic skin response); blood pressure; sleep patterns; activity level; mood; stress level; geographic location; a therapy session transcript; textual patient assessment; qualitative patient assessment; and/or quantitative patient assessment. The patient data may include data from a patient application such as chats, support groups, posts or daily check-ins and surveys, as shown in FIG. 3 The patient data may include data from a therapist application such as questionnaires and patient intakes, meeting summaries (e.g. therapist notes, transcripts), medical impressions, and/or relapse history, as shown in FIG. 3.

In some embodiments, method 400 includes preprocessing the patient data. For example, the preprocessing may include one or more of: natural language processing (NLP); named entity detection (NER); and/or tokenization.

Method 400 may include predicting, by a trained neural network model, based on the patient data, an addiction relapse risk in the patient (Step 420). The neural network model may be trained on historic patient data from one or multiple patients. The neural network may be trained using techniques known in the art or otherwise as described herein. The neural network may undergo continuous learning in order to be trained (e.g. further trained) on more recent patient data, such as real-time patient data. The neural network model may be, or may include, a relapse risk predictive model, such as relapse risk predictive model 223 or 323 shown in FIGS. 2 and 3, respectively.

An addiction relapse risk may be a score, categorization, probability, or other indicator of a risk of relapse in a patient. In some embodiments, the addiction relapse risk is categorized as none, low, or high.

Method 400 may include generating a recommendation based on the predicted addiction relapse risk (Step 430). A recommendation may be a suggested action or intervention for the patient. A recommendation related to a low risk may be different to a recommendation relating to a high risk.

In some embodiments, generating a recommendation based on the predicted addiction relapse risk includes generating the recommendation using generative artificial intelligence. For example, a generative AI module such as generative AI model 226 may be used. The generative AI may be a large language model (LLM) trained to receive an input prompt and generate an output (such as text or an image) based on the prompt.

In some embodiments, the recommendation includes one or more of: a request for additional patient data; an intervention; a stress management technique; and/or a personalized message.

Method 400 may include communicating the recommendation to a relevant party (Step 440). For example the recommendation may be sent to a device (such as a mobile, laptop, smart watch or other personal device) of the relevant party. A relevant party may include the patient and/or the patient's therapist, but may also include: one or more other therapists (e.g. as part of a therapy team for the patient); a predefined friend of the patient; a predefined family member of the patient; a caregiver of the patient; a sponsor of the patient;

a care worker; a social worker; a support worker; a medical professional; and/or an emergency service (e.g. 911, ambulance), or the like.

In some embodiments, if the addiction relapse risk is categorized as low, method 400 may include communicating the recommendation to the patient, wherein the recommendation includes a request for additional patient data. For example, a patient application may provide a push notification requesting more data from the patient via a check-in or survey.

In some embodiments, if the addiction relapse risk is categorized as high, the method may include communicating the recommendation to the patient and a therapist of the patient, wherein the recommendation includes a suggested intervention generated by a generative artificial intelligence, and wherein the suggested intervention optionally includes input from the therapist. For example, a generative AI may provide an initial intervention strategy, which may be supplemented by the therapist based on the therapist's knowledge, experience, and/or working relationship with the patient. This may decrease a therapist's workload for an individual patient and may allow for the therapist to work with a greater number of patients, extending the capabilities of the therapist. In some embodiments, a suggested intervention may be sent automatically to the patient.

An intervention may include an enforcement which may be carried out by embodiments of the invention. For example, an enforcement may be or may relate to one or more of: Creating geofencing based on the user's location; automatically connect emergency service or contact person; providing incentives to encourage positive behavior (e.g. positive reinforcement); and/or real-time biofeedback intervention, as described herein.

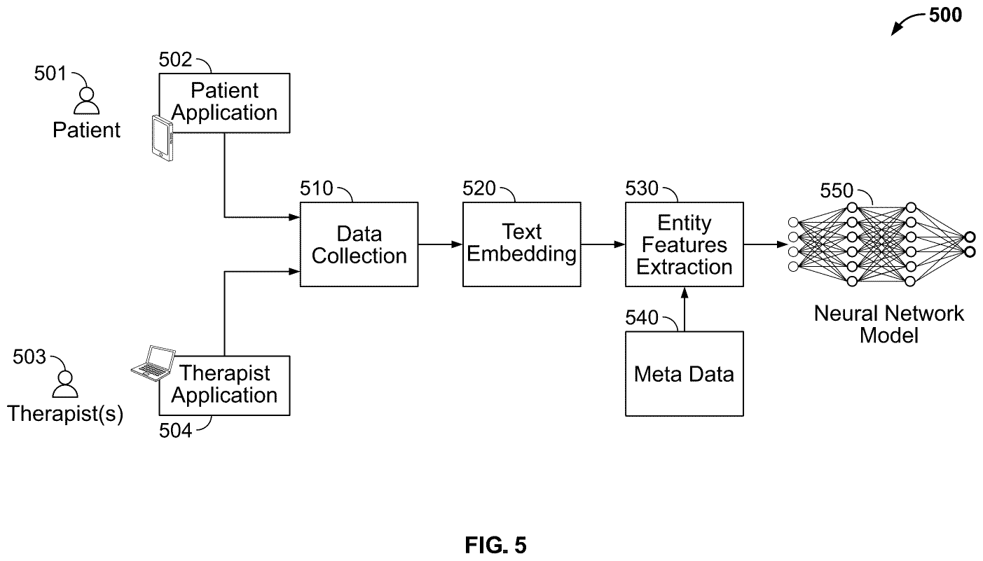

FIG. 5 is a schematic flow diagram of an example processing of user input, according to some embodiments of the invention. In the flow 500 shown in FIG. 5, a patient 501 using patient application 502, and therapist 503 using therapist application 504 provide data (e.g. actively or passively) to a data collection process 510. Flow 500 may include text embedding 520, which include, for example, tokenization of textual data which is converted into a dense vector representation. Each token may be mapped to a vector in a high-dimensional space, where the location and distance (e.g. determined by a norm or metric) between vectors capture semantic meaning.

Flow 500 may include entity feature extraction 530, which may include the use of metadata 540 to extract relevant features, such as named entities, indicative physiological parameters, contextual messages (e.g. that the patient is due to attend a stressful event), or the like. Data reduction techniques may be used as part of feature extraction.

Flow 500 may include inputting pre-processed data (e.g. collected patient data which has undergone text embedding and entity feature extraction) to a neural network model 550, such as a relapse risk predictive model. Neural network model 550 may be an artificial neural network (ANN), convolutional neural network (CNN), a recurrent neural network, a feed-forward neural network, or other type of neural network. neural network model 550 may include an input layer, one or more hidden/intermediate layers, and an output layer. The output may be a prediction of an addiction relapse risk in the patient, for example as a category (e.g. none, low, high) or as a probability.

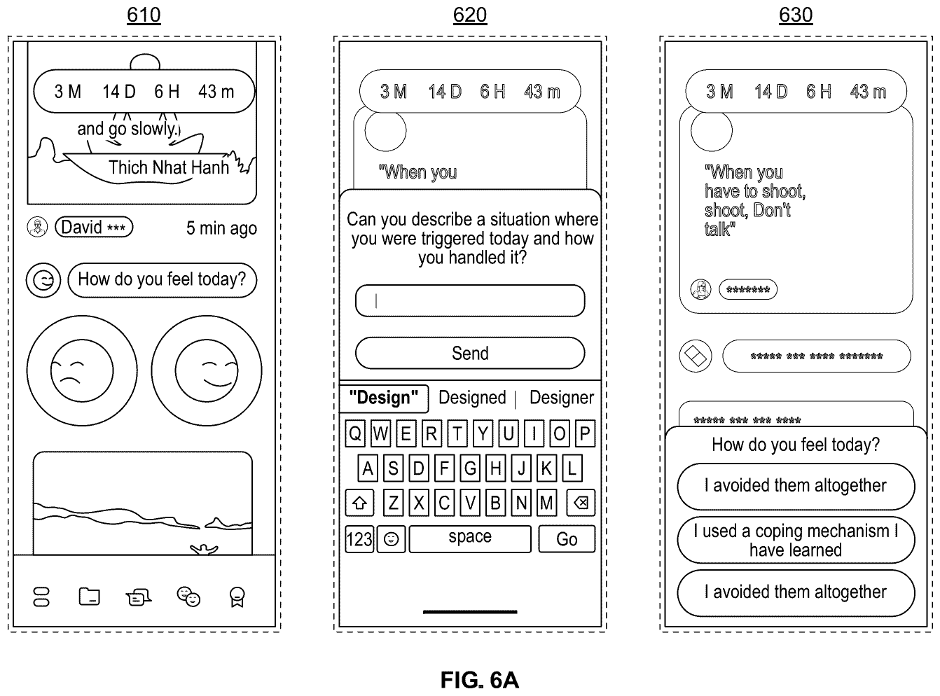

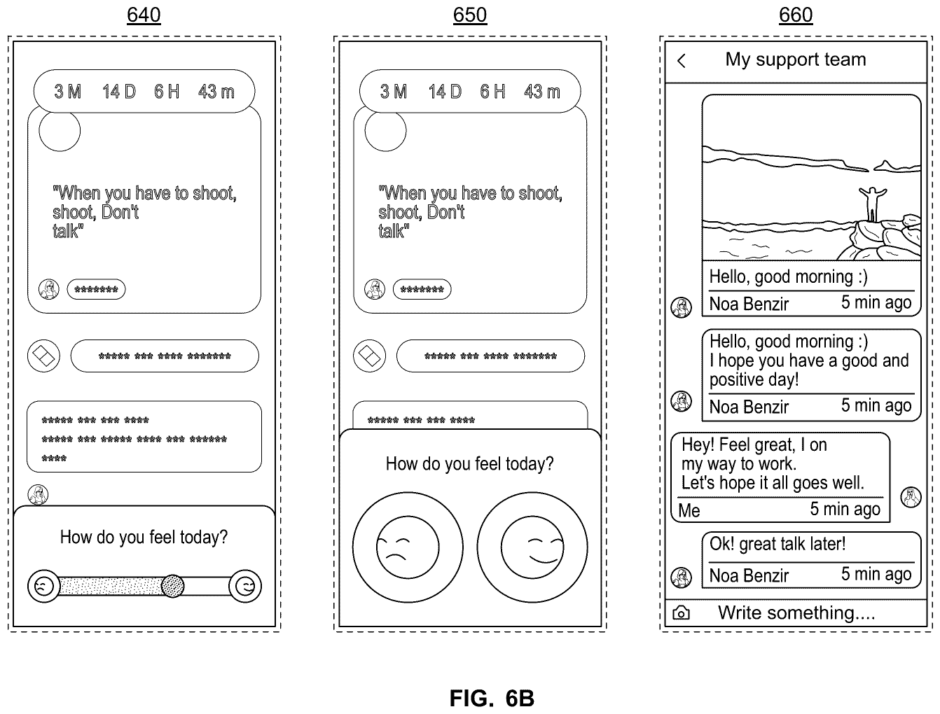

FIGS. 6A and 6B illustrate example views of a patient mobile device application for collecting data, according to some embodiments of the invention. For example, patient application 202/302/502 shown in FIG. 2/FIG. 3/FIG. 5 may include one or more of the views shown in FIGS. 6A and 6B.

For example, as shown in view 610 (and 650), the patient application may collect an assessment of the patient's mood on the main feed of the application (app). A question such as “How are you feeling today?” may be accompanied by an opportunity for patient input, such as an interactable (e.g. via touch screen) happy face or sad face allowing the patient to convey their emotional state.

In some embodiments, a patient may provide a written textual assessment of their mood, for example by typing in a free-text box of the application, as shown in view 620. A prompting question which the app displays to the user may be an open-ended question, which may allow a better insight into the individual state of the patient using NLP and NER techniques described herein.

In some embodiments, a patient may be presented with a number of predefined responses to a question, such as is shown in view 630. This type of data collection may be used when more specific patient data is required at an asynchronous time, for example because there was a predicted low risk of addiction relapse (e.g. Step 352 of FIG. 3).

In some embodiments, a patient may provide a rating on a scale, such as is shown in view 640.

A patient application in accordance with embodiments of the invention may include a chat functionality, for example as shown in view 660, where a patient can send and receive messages to/from their support team, such as a therapist. The messages from the support team may include daily motivational content. Messages from the patient may be analyzed using NLP and NER techniques described herein in order to provide patient data.

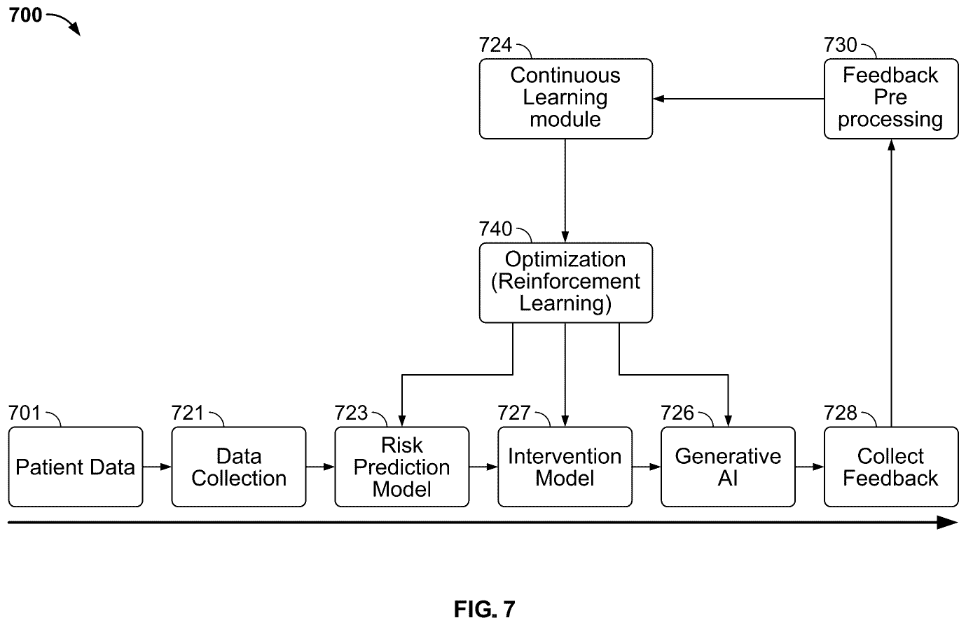

FIG. 7 is a schematic flow diagram of an example model integration and processing flow, according to some embodiments of the invention. Flow 700 shown in FIG. 7 may include patient data 701 collected by a data collection process/module 721. the data may be preprocessed. The data may be input to a risk prediction model 723, which may be a neural network. Based on the predicted risk of relapse, an intervention model 727 may generate an intervention strategy, which may be supplemented by tailored recommendations by generative AI 726.

Flow 700 may include the collection of feedback 728, for example reviews or assessments of how useful/accurate the predictions and recommendations are. Feedback may be processed 730, which may be utilised as part of continuous learning 724 for improving the predictive models. Optimization 740 may include reinforcement learning techniques to improve risk prediction model 723, intervention model 727, and/or generative AI 726.

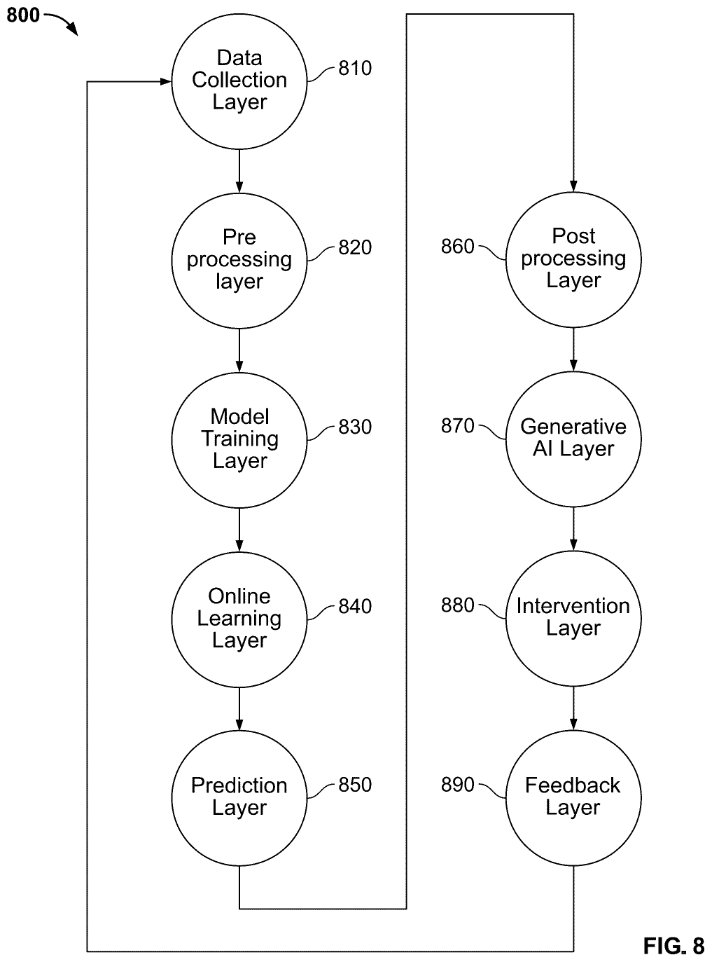

FIG. 8 shows example processing layers, according to some embodiments of the invention. The processing layers may be arranged in a structure 800. Structure 800 may include a data collection layer 810, a preprocessing layer 820, a model training layer 830, an online learning layer 840, a prediction layer 850, a postprocessing layer 860, a generative AI layer 870, an intervention layer 880, and/or a feedback layer 890.

Data collection layer 810 may be responsible for collecting new data from users (e.g. patients and/or therapists). This could involve monitoring user activity on a mobile app (e.g. patient application and/or therapist applications described herein), collecting user responses to surveys, and/or tracking biometric data from wearable devices. The data may be collected in a privacy preserving manner with user consent.

Preprocessing layer 820 may preprocess data collected by data collection layer 810 to prepare the data for a deep learning/machine learning model according to embodiments of the invention. Preprocessing may include cleaning the data, handling missing values, normalizing numerical data, and/or encoding categorical data.

Model training layer 830 may include a deep learning model. This layer may combine deep learning algorithms such as convolutional neural networks for image data, recurrent neural networks for time-series data, and a transformer-based model for text data.

Online learning layer 840 may enable continuous learning by retraining the model regularly with newly collected data (e.g. patient data collected by data collection layer 810). This may involve fine-tuning the existing model on the new data, or retraining the model from scratch if enough new data has been collected. The fine-tuning may include modifying one or more weights or biases associated with connections between neurons in the one or more combined neural networks.

Prediction layer 850 may utilize the trained model (e.g. layer 830) to make predictions on new data. For example, predicting if a user is at risk of relapse based on their recent activity and interactions.

Postprocess layer 860 may interpret the results of prediction layer 850 and prepare the result for eventual submission to intervention layer 880. This may involve converting the model's output into a probability score, or grouping different predictions into categories.

Generative AI layer 870 may, based on the interpreted results from postprocessing layer 860 and other data such as the user's history, preferences, and characteristics, generate personalized content for intervention. This content may include messages, recommendations, strategies, or any other suitable technique.

Intervention layer 880 may include a decision process to decide on an appropriate intervention and may execute the chosen intervention. This may include sending a notification to the patient's mobile device, scheduling a session with a therapist, alerting a relevant party such as a family member, friend, sponsor, or the like, or any other suitable action.

Feedback layer 890 may involve inputting the results of the intervention back into the system, allowing the model structure 800 to learn from its actions and improve over time. This may involve collecting user feedback on the intervention (e.g. feedback from patients and/or therapists) and tracking whether the user's behavior changed following the intervention.

According to one or more embodiments of the invention there is provided a system. The system may include at least one computer processor; and a memory containing instructions which, when executed by the at least one computer processor, cause the at least one computer processor to carry out methods described herein. For example, the at least one computer processor may: receive patient data of a patient; execute a neural network model trained to predict, based on the patient data, an addiction relapse risk in the patient; generate a recommendation based on the predicted addiction relapse risk; and communicate the recommendation to a relevant party.

The computer processor and/or memory may be part of a computing device, such as computing device 100 shown in FIG. 1.

As described herein, the patient data may be received from one or more of: a wearable device; a mobile device; a mobile application; and/or a telemedicine system. The patient data may include one or more of: heart rate; heart rate variability; skin conductance; blood pressure; sleep patterns; activity level; mood; stress level; geographic location; a therapy session transcript; textual patient assessment; qualitative patient assessment; and/or quantitative patient assessment.

In some embodiments, the at least one computer processor is configured to preprocess the patient data, wherein the preprocessing includes one or more of: natural language processing (NLP); named entity detection (NER); and/or tokenization, as described herein.

The at least one computer processor may be configured to generate a recommendation based on the predicted addiction relapse risk by generating the recommendation using generative artificial intelligence. The recommendation may include one or more of: a request for additional patient data; an intervention; a stress management technique; and/or a personalized message.

In some embodiments, the at least one computer processor is configured to categorize the addiction relapse risk as none, low, or high, as described herein.

If the addiction relapse risk is categorized as low, the at least one computer processor may be configured to communicate the recommendation to the patient, wherein the recommendation includes a request for additional patient data, as described herein.

If the addiction relapse risk is categorized as high, the at least one computer processor may be configured to communicate the recommendation to the patient and a therapist of the patient, wherein the recommendation includes a suggested intervention generated by a generative artificial intelligence, and wherein the suggested intervention optionally includes input from the therapist, as described herein.

According to one or more embodiments there is provided a method for predicting relapse in a patient. The method may include training a neural network model on patient data relating to a plurality of patients. For example, the patient data may be historic data of a large body of patients (e.g. hundreds to thousands of patients). The neural network may be trained according to one or more techniques known in the art or otherwise described herein.

The method may include predicting, by the trained neural network model, based on a set of runtime patient data, a relapse risk of the patient. The runtime patient data may be real-time data collected from a particular patient of interest. Real-time may correspond to an event to system response on the order of milliseconds or microseconds. The relapse risk may be a score, probability, or classification of a risk of addiction relapse in patient, as described herein.

The patient data relating to the plurality of patients and/or the runtime patient data may be collected from one or more of: a wearable device; a mobile device; a mobile application; and/or a telemedicine system. The patient data relating to the plurality of patients and/or the runtime patient data may include one or more of: heart rate; heart rate variability; skin conductance; blood pressure; sleep patterns; activity level; mood; stress level; geographic location; a therapy session transcript; textual patient assessment; qualitative patient assessment; and/or quantitative patient assessment, as described herein.

The method may include recommending an action based on the predicted relapse risk. For example, the action may be the sending of a motivational message, a stress management technique such as a breathing exercise, an intervention strategy, or the like. Recommending an action based on the predicted relapse risk may include generating the recommended action using generative artificial intelligence, for example, a motivational message may be generated by the artificial intelligence and tailored to the particular patient for which it is intended.

The method may include notifying the patient or a predefined contact of the predicted relapse risk and recommended action. For example, the patient may receive a stress management technique, a family member may receive an intervention strategy for the patient, or the like.

An intervention may include an enforcement which may be carried out by embodiments of the invention. For example, an enforcement may be or may relate to one or more of: Creating geofencing based on the user's location; automatically connect emergency service or contact person; providing incentives to encourage positive behavior (e.g. positive reinforcement); and/or real-time biofeedback intervention.

In some embodiments, the method includes preprocessing the patient data relating to the plurality of patients and/or the runtime patient data. The preprocessing may include one or more of: natural language processing (NLP); named entity detection (NER); and/or tokenization, as described herein.

The preprocessing may be used to extract features relevant to relapse risk contexts and correspondingly label the patient data relating to the plurality of patients for training the neural network model. For example, patient triggers may be identified from patient messages, which may be labelled as a trigger for the training of the neural network in order to improve prediction relapse risk.

The model may utilize machine learning methods to optimize prediction accuracy:

Deep learning—This may involve creating a neural network with multiple layers that can learn to recognize complex relapse patterns in the data. For example, a recurrent neural network (RNN) and long short-term memory (LSTM) models are used to analyze a time-series data from the patient's relapse history triggers, in combination with a convolutional neural network (CNN) that is used for processing the textual data from summaries and patient's inputs.

Unsupervised Learning—These techniques may be used for clustering groups of patients with similar characteristics or behaviors. This technique may provide insights into different risk profiles for relapse and inform more tailored interventions.

Reinforcement learning—This may be used to optimize the timing and content of interventions. This model learns to make decisions, for example, when to send an alert or suggest and intervention, based on the goal of maximizing the reward signal which is avoidance of relapse (e.g. the longest continuous term of sobriety from the addictive behavior or substance).

Continually updating neural network models according to embodiments of the invention with new data from the individual can allow it to adapt over time. For example, the model could learn that certain physiological indicators or behavioral patterns are particularly predictive of relapse for a specific individual and adjust its predictions accordingly.

By integrating and interconnecting AI algorithms, including Natural Language Processing (NLP), Machine Learning (ML), and/or Behavioral Psychology principles, embodiments of the invention use data driven predictive algorithms that may intelligently predict what data would be most useful to collect from the user at a given point of time. This method can increase the effectiveness and efficacy of the system predictions and interventions.

### Initial Data Collection

The system may collect baseline data from each user. This could involve demographics, addiction history, lifestyle factors, and/or other relevant information. The data can be collected through surveys, intakes, interviews, direct input on a mobile connected device and/or from wearable devices.

### Prediction Algorithm Driven Data Collection

The predictive model may output a risk of relapse score. This score may be categorized based on threshold into three categories-none, low, and high. Other categories and/or additional categories may be used. If the prediction risk of relapse level is low, then additional data may be useful. For example, if the system predicts that the user is at low risk of relapse, it might trigger a request for more information about their current emotional state or recent substance use by triggering more daily check-ins such as asking the patient how they feel.

### Triggered Data Collection

The predictive algorithm can drive a triggered data collection process. For example, the algorithm could use the user's input, time, location and other context data to predict when it might be most useful to prompt for additional data. For example, if a patient who is addicted to gambling is in close proximity to a place that provides sports gambling services, then the system can trigger a data collection via a check-in for personal mode, or may ask the patient directly if they feel that they are close to a relapse. The trigger can be done via push notification on the user's mobile device, wearable device or any other type of connected device.

### Adaptive Data Collection

Over time, the system may use algorithms to adapt its data collection strategy based on the usefulness of the data collected so far and any feedback provided by the user. For example, if the system finds that a particular type of data is particularly predictive for a certain user, it will prioritize collecting more of that type of data. Conversely, if a user frequently ignores or rejects requests for a certain type of data, the system may deprioritize those requests. For adaptive data collection the system can use an RL (reinforcement learning) algorithm. The state of the RL algorithm may be defined by the current state and condition of the patient, which can include the latest information about the patient, including chat interactions, self-reports, and other available data. The actions that the RL algorithm can take could be different types of data collection requests that the system sends to the patient, for example, asking the user to answer a survey or initiate a chat interaction. An important part of the RL algorithm may be the reward signal which indicates the effectiveness of an action to collect data.

A reward could be given when the requested data leads to useful prediction of risk of relapse. If the user rejects the request, or ignores the request, this could be considered a negative reward or penalty for the RL algorithm. The objective of the RL algorithm may be to maximize the cumulative reward over time. For example, the RL algorithm may seek to make the most accurate predictions and intervention possible, based on the most relevant data.

A value function may be defined, which may be the prediction of the future reward that can be obtained from a given state or a state action pair. The RL algorithm may start by exploring the environment, for example, by sending different types of data collection request to the patients and observing the outcomes. Based on these experiences the RL algorithm may update its policy-which defines the behavior of each state- and the value function, gradually learning which actions are the most effective in which state. This process may continue indefinitely, with the agent constantly adapting and improving its strategies based on the ongoing experiences and the changing condition of the patient. This way, the RL algorithm can learn an optimal adaptive data collection strategy that balances the need for accurate and timely predictions and interventions with the need for patient engagement and data availability.

### Real-Time Updating

Collected data may be used to update the system's predictive model in real-time, making it constantly adaptive and improving its understanding of the user's condition and needs. This continual learning process may allow the system to become more effective over time. For example, the model may continuously learn and adapt based on new data as it is collected, rather than being periodically retrained on a static dataset. This may allow the model to immediately incorporate new insights from the latest data, enhancing its performance and its understanding of the user's condition and needs over time. As the patient interacts with the system, new data may be constantly being generated. This can include the patient's responses to questions, their engagement with recommended interventions, physiological data from wearable devices, and so on. The collected data may be preprocessed and transformed into a format that the AI model can understand. For text data, this could involve tokenization (breaking text down into individual words or sub-words), removing stop words (common words like ‘and’, ‘the’, ‘is’ that usually carry little meaningful information), and encoding (representing words as numerical vectors). For physiological data, preprocessing might involve normalizing values or identifying relevant features like heart rate variability or sleep patterns. The processed data may be used to update the parameters of the model. This may be done using a machine learning algorithm that incrementally adjusts the model's parameters based on the new data. The updated model may then be used to predict the patient's risk of relapse and recommend personalized interventions. Since the model has been updated with the latest data, these predictions and recommendations are based on the most up-to-date understanding of the patient's condition and needs.

The decision support module may generate personalized recommendations or interventions based on the predicted relapse risk. These may include alerts to the individual or their support network, suggestions for coping strategies or stress management techniques, or adjustments to the treatment plan as deemed appropriate by healthcare professionals. Personalized Intervention Recommendation

The AI model examines the patient's input text, and—if available—physiological data, and other relevant variables such as frequency of therapy sessions, social interactions. Each of these factors contribute to the risk score that the model generates. The computation of the risk score may be a probabilistic prediction of the likelihood of a relapse. The threshold which constitutes a “high risk” may be set according to clinical expertise or statistical analysis. For instance, one might set a rule that if the model predicts a score greater than 50% chance of relapse within the next week, that is considered a high risk. The urgency of intervention may similarly be determined by the risk score and potentially other factors. For example, a rapid increase in the risk score might indicate an urgent need for intervention, even if the absolute risk level is still below the high-risk threshold. Additionally, certain triggers or symptoms could be programed into the model as immediate red flags for intervention, regardless of the overall risk score. For instance, if a patient reports using their substance, or experiencing sever withdrawal symptoms, that may necessitate immediate intervention.

Based on this predictive algorithm, if the system predicts a high-risk of relapse or identifies an urgent need for intervention then it may recommend a specific intervention based on the user's personal data and circumstances. For example, if the user's data suggest that they are feeling stressed and they've previously reported that stress is a major trigger for their substance use, the system may recommend a stress management technique, such as deep breathing exercises, mindfulness meditation, or taking a walk outside.

The system may send these recommendations to the therapist or to the user in real-time via push notifications on the mobile device or other connected device.

In some embodiments, the system may also initiate automated interventions. For example, it can schedule a virtual meeting with a therapist, share inspirational content, or reminders about the user's recovery goals, or connect the user with a supportive community of other individuals who are also in recovery.

The evaluation module may continuously assess the performance of the neural network model and update the model parameters based on feedback from the decision support module. This iterative process may facilitate continuous learning and improvement. This may be done as the user continues to interact with the system, for example collecting more data from various sources (e.g. chat interactions, user reported information, etc.). Each of these interactions may give the system more content and richer understanding of the patient's current state, patterns of behavior, triggers, and responses to interventions. The system may use incremental learning by updating its models periodically based on the newly acquired data. This could be done through online learning, where the model is updated after each new piece of data, or alternatively through batch learning where the model is updated after collecting a certain amount of new data. After each update, the model's performance may be evaluated to ensure that it is improving. The model evaluation may be done using several key methods such as Confusion matrix, Cross validation, Holdout validation and ROC Curve and AUC Score.

### Personalized Intervention Based on Generative AI

A generative AI model (e.g. a GPT) may generate personalized recommendations and/or interventions in real time by leveraging the advanced natural language processing capabilities of the generative AI model. The module may be pre-trained on a large corpus of text data from various sources, such as addiction treatment guidelines, therapy transcripts, or self-help resources, to ensure the generated interventions are contextually relevant, evidence-based, and linguistically coherent.

Generative AI models are a type of machine learning model that can generate new, original content. They do this by learning the underlying patterns and structures in the data. In the context of addiction recovery support, the system generative AI model may be trained on diverse corpus of addiction related text, allowing it to generate personalized recommendations or interventions. This could include transcripts of therapy sessions, addiction treatment guidelines, scientific literature on addiction, self-help books, and more. The model may learn to predict the next word in a sentence given the previous words. Through this process, it learns not just vocabulary or grammar, but also the types of ideas and structures that are common in addiction recovery data. After the initial training, the model may be further fine-tuned on more specific datasets that are closely related to the task of addiction recovery support. For example, the model may be fine-tuned on a dataset of supportive messages for patients who are in the process of addiction recovery. Once the model has been trained and fine-tuned, it can generate new content. This can be done by providing the model with a ‘prompt’, which could be a few sentences that describes a patient's current situation. The model may then generate text that continues the prompt, based on the patterns it learned during training.

For example, the user can write a message on the support chat in the mobile device application: “I'm feeling really stressed and I'm worried”. This input may be processed by the data collection model, and then its output is sent as an input to the risk prediction model. The risk prediction model assesses the risk for relapse. Then, if the risk high, the system may collect all the patient's data and create an example prompt for the generative AI model such as “create a support message for a male, age 35, of a medium socioeconomic background, addicted to alcohol, and with a high-risk for immediate relapse”. The model might generate a response such as: “I'm really sorry that you're feeling this way, but remember, it's just a temporary state. Can you try to engage in a calming activity, like taking a deep breath or going for a walk? It could also be helpful to reach out to a supportive friend or family member. Remember, it's okay to ask for help.”

This generated response resembles the type of advice that a trained professional might give and is personalized to the user's current situation. However, it is generated in real-time by the AI model, making it scalable and accessible at any time, unlike with a therapist, who may not be available at the crucial time a patient is at risk of relapse (such as late at night), or who may have a high caseload and cannot work on the patient's case until after dealing with other patients. This message could also be sent on behalf of the social-worker or therapist, allowing more engaging experience for the patient and at the same time provide more scale and efficiency for the professional caregivers.

The evaluation module may continuously assess the performance of the neural network model and generative AI based intervention module and update the model parameters based on feedback from the decision support module. This iterative process may facilitate continuous learning and improvement, ensuring that the algorithm remains effective and adapts to the changing needs of the individual.

In some embodiments, a neural network algorithm with real-time generative AI for interventions may be integrated into a telemedicine system, enabling remote monitoring and support for individuals undergoing addiction treatment. The telemedicine system may include a secure platform for data sharing and communication between the individual, healthcare professionals, and caregivers. This may facilitate real-time collaboration and decision-making, as well as improve access to care for individuals in remote or underserved areas.

The generative AI may analyze the user's data to understand their unique needs and circumstances. This may include their demographic information, addiction history, triggers, stressors, lifestyle, preferences, and other relevant factors. Using these insights, the generative AI may create personalized content for the user. For example, the content can include motivational messages, suggestions for healthy coping strategies, reminders about the user's recovery goals, and/or educational content about addiction and recovery. The content may be tailored to the user's unique needs and circumstances and written in a style that is likely to resonate with them.

Accordingly, the present invention may provide a neural network algorithm with real-time generative AI intervention for predicting relapse in individuals undergoing addiction treatment and generating personalized interventions. By leveraging personal data and advanced natural language processing techniques, embodiments of the invention have the potential to significantly improve addiction treatment outcomes and support long-term recovery.

A decision support module may communicate the generated personalized content, recommendations or interventions to the individual, healthcare professionals, caregivers, or other relevant parties for timely action. These interventions may include alerts to the individual or their support network, suggestions for coping strategies or stress management techniques, or adjustments to the treatment plan as deemed appropriate by healthcare professionals.

The decision support module may provide insights into the user's behavior, mood changes, triggers and risk factors that can help the therapist to better understand their patient's unique challenges and needs. The AI algorithm may also perform risk prediction that can alert the therapist to potential crises before they occur, allowing them to proactively adjust their treatment plan or schedule additional sessions if necessary.

The system can evaluate the effectiveness of different strategies by analyzing changes in the user's risk level, mood or another relevant factor following different interventions. These insights may help therapists identify what strategies are most effective for that individual and may use the generative AI to create personalized treatment plans that maximize the chances of a successful recovery.

The AI system may provide continuous monitoring and support between therapy sessions, enhancing the continuity of care and enabling therapists to stay informed about their patient's progress even when they are not in session.

By automating aspects of data collection and analysis, embodiments of the invention may save therapist time and allow them to focus on more direct patient care, and also increase the number of patients they are capable of providing care services to. Embodiments may facilitate better communication and engagement between the patient and the therapist. Patients may be more likely to communicate their feelings, cravings, or triggers using the system, and this information may be relayed to the therapist.

Embodiments of the invention may be designed to be beneficial to all the stakeholders involved in the support and treatment of patients who suffers from addiction, among them:

Social Workers-social workers often play a crucial helping individuals navigate the complexities of addiction recovery, including accessing necessary resources and services. Embodiments may provide social workers with data-driven insights about the individual's needs and challenges, allowing them to more effectively coordinate services and advocate for the insights. Embodiments may also alert social workers when an individual is at a high risk of relapse, and provide personalized suggestion for intervention.

Psychologists-Psychologists may provide vital therapeutic support to individuals in recovery. The AI algorithms of embodiments of the invention may enhance this support by providing the psychologist with additional information about the individual mood, stress levels, triggers and other relevant factors. Embodiments may predict relapse risk and help the psychologist adjust their therapeutic strategies. Furthermore, data collected by embodiments of the invention may help psychologists to evaluate the effectiveness of different therapeutic interventions and to personalize their approach and intervention based on what works best for the individual.

Sponsors—In programs like Alcoholics Anonymous (AA), sponsors provide personal support and mentorship to individuals in recovery. Embodiments of the invention may help sponsors by alerting them when the individual they are sponsoring is at high risk of relapse, enabling them to provide additional support at critical times. Embodiments of the invention may provide sponsors with information about what strategies or coping mechanisms have been most effective for the individual, enabling them to provide more targeted advice and guidance.

Family members and close Friends-Family members and close friends may provide emotional and physical support. Embodiments of the invention may share information in real-time about their relative or friend in recovery process, and may provide them with personalized content that help them to cope and understand the situation of their family member/friend.

Management and policymakers-Embodiments of the invention may provide consolidated, data-driven analysis about addiction behaviors and trends. Embodiments may help policymakers make informed decisions about where to allocate resources, what interventions to prioritize, and what policies are likely to be most effective in supporting recovery. For the management, embodiments may be used to monitor the performance of therapists, social workers, and other professionals involved in supporting recovery.

Embodiments of the invention may identify circumstances which may result in a relapse. In the event of substance related relapses, embodiments of the invention may provide an early warning, thereby reducing risk of personal harm, injury, or death. For example, in the case of recovering opioid patients, the patient's body may no longer be accustomed to their historic dose, and thus a patient who relapses and attempts to take their historic dose may be at a risk of overdose, which may result in death. Embodiments of the invention may prevent this.

Additionally, embodiments of the invention may extend the capabilities of therapists, allowing therapists to handle more patients and reduce waiting times to see a therapist.

As used herein, a machine learning model may include a neural network and may receive input data. The input data may be, for example, an image. A machine learning model may output predictions calculated, estimated, or derived on the basis of function approximation and/or regression analysis. A neural network may include neurons or nodes organized into layers, with links between neurons transferring output between neurons. A neural network may include an input layer, one or more hidden layers, and an output layer. Aspects of a neural network may be weighted, e.g. links may have weights and/or biases, and training may involve adjusting weights and/or biases. A positive weight may indicate an excitatory connection, and a negative weight may indicate an inhibitory connection. A neural network may be executed and represented as formulas or relationships among nodes or neurons, such that the neurons, nodes, or links are “virtual”, represented by software and formulas, where training or executing a neural network is performed, for example, by a conventional computer or GPU (such as computing device 100 in FIG. 1). A machine learning based predictive model may be a reinforcement learning based model. Reinforcement learning algorithms may be based on dynamic programming techniques and may include using a Markov decision process (MDP) such as a discrete-time stochastic control process. Reinforcement learning models may be advantageous over supervised learning models because they do not require labelled input data and may be used where constructing an exact mathematical model is infeasible.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented. and embodiments not specifically described may include various features described herein.

1. A method comprising:
receiving patient data of a patient;
predicting, by a trained neural network model, based on the patient data, an addiction relapse risk in the patient;
generating a recommendation based on the predicted addiction relapse risk; and
communicating the recommendation to a relevant party.

2. The method of claim 1, wherein the patient data is received from one or more of:
a wearable device; a mobile device; a mobile application; and a telemedicine system.

3. The method of claim 1, wherein the patient data comprises one or more of:
heart rate; heart rate variability; skin conductance; blood pressure; sleep patterns; activity level; mood; stress level; geographic location; a therapy session transcript; textual patient assessment; qualitative patient assessment; and quantitative patient assessment.

4. The method of claim 1, comprising preprocessing the patient data, wherein the preprocessing comprises one or more of:
natural language processing (NLP); named entity detection (NER); and tokenization.

5. The method of claim 1, wherein generating a recommendation based on the predicted addiction relapse risk comprises generating the recommendation using generative artificial intelligence.

6. The method of claim 1, wherein the recommendation comprises one or more of:
a request for additional patient data; an intervention; a stress management technique; and a personalized message.

7. The method of claim 1, wherein the addiction relapse risk is categorized as none, low, or high,
wherein if the addiction relapse risk is categorized as low, the method comprises communicating the recommendation to the patient, wherein the recommendation comprises a request for additional patient data, and
wherein if the addiction relapse risk is categorized as high, the method comprises communicating the recommendation to the patient and a therapist of the patient, wherein the recommendation comprises a suggested intervention generated by a generative artificial intelligence, and wherein the suggested intervention optionally comprises input from the therapist.

8. A system comprising:
at least one computer processor; and
a memory containing instructions which, when executed by the at least one computer processor, cause the at least one computer processor to:
receive patient data of a patient;
execute a neural network model trained to predict, based on the patient data, an addiction relapse risk in the patient;
generate a recommendation based on the predicted addiction relapse risk; and
communicate the recommendation to a relevant party.

9. The system of claim 8, wherein the patient data is received from one or more of:
a wearable device; a mobile device; a mobile application; and a telemedicine system.

10. The system of claim 8, wherein the patient data comprises one or more of:
heart rate; heart rate variability; skin conductance; blood pressure; sleep patterns;
activity level; mood; stress level; geographic location; a therapy session transcript; textual patient assessment; qualitative patient assessment; and quantitative patient assessment.

11. The system of claim 8, wherein the at least one computer processor is configured to preprocess the patient data, wherein the preprocessing comprises one or more of:
natural language processing (NLP); named entity detection (NER); and tokenization.

12. The system of claim 8, wherein the at least one computer processor is configured to generate a recommendation based on the predicted addiction relapse risk by generating the recommendation using generative artificial intelligence.

13. The system of claim 8, wherein the recommendation comprises one or more of:
a request for additional patient data; an intervention; a stress management technique; and a personalized message.

14. The system of claim 8, wherein the at least one computer processor is configured to categorize the addiction relapse risk as none, low, or high,
wherein if the addiction relapse risk is categorized as low, the at least one computer processor is configured to communicate the recommendation to the patient,
wherein the recommendation comprises a request for additional patient data, and wherein if the addiction relapse risk is categorized as high, the at least one computer processor is configured to communicate the recommendation to the patient and a therapist of the patient, wherein the recommendation comprises a suggested intervention generated by a generative artificial intelligence, and wherein the suggested intervention optionally comprises input from the therapist.

15. A method for predicting relapse in a patient, the method comprising:
training a neural network model on patient data relating to a plurality of patients;
predicting, by the trained neural network model, based on a set of runtime patient data, a relapse risk of the patient;
recommending an action based on the predicted relapse risk; and
notifying the patient or a predefined contact of the predicted relapse risk and recommended action.

16. The method of claim 15, wherein the patient data relating to the plurality of patients and the runtime patient data are collected from one or more of:
a wearable device; a mobile device; a mobile application; and a telemedicine system.

17. The method of claim 15, wherein the patient data relating to the plurality of patients and the runtime patient data comprises one or more of:
heart rate; heart rate variability; skin conductance; blood pressure; sleep patterns; activity level; mood; stress level; geographic location; a therapy session transcript; textual patient assessment; qualitative patient assessment; and quantitative patient assessment.

18. The method of claim 15, comprising preprocessing the patient data relating to the plurality of patients and the runtime patient data, wherein the preprocessing comprises one or more of:
natural language processing (NLP); named entity detection (NER); and tokenization.

19. The method of claim 18, wherein the preprocessing is used to extract features relevant to relapse risk contexts and correspondingly label the patient data relating to the plurality of patients for training the neural network model.

20. The method of claim 1, wherein recommending an action based on the predicted relapse risk comprises generating the recommended action using generative artificial intelligence.
